(19) United States
(12) Patent Application Publication
(10) Pub. No.: US 2009/0254061 A1
(43) Pub. Date: Oct. 8, 2009

(54) METHOD AND APPARATUS FOR REMOVING CEREBROSPINAL FLUID

(75) Inventor: Eric Peter Baron, Broadview Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/416,986

(22) Filed: Apr. 2, 2009

Related U.S. Application Data
(60) Provisional application No. 61/041,849, filed on Apr. 2, 2008.

(51) Int. Cl. A61B 17/34; A61M 1/00

(52) U.S. Cl. 604/506; 604/173

(57) ABSTRACT

An apparatus for removing cerebrospinal fluid includes a first needle having a first end, a second end, and a passage extending between the first and second ends. The proximal end of the first needle has a handle and the distal end has a cutting tip adapted to penetrate skin. A second needle is slidably disposed within the passage of the first needle. The second needle has a proximal end, a distal end, and a lumen extending between the proximal and distal ends. The proximal end of the second needle has a handle. The distal end of the second needle has an atraumatic tip and a microfilter in fluid communication with the lumen for removing cerebrospinal fluid.

### RELATED APPLICATION

The present application claims priority from U.S. Provisional Application No. 61/041,849, filed Apr. 2, 2008 herein incorporated by reference in its entirety.

### TECHNICAL FIELD

The present invention is directed to a method and apparatus for removing cerebrospinal fluid.

### BACKGROUND OF THE INVENTION

The use of a needle to puncture the skin of a patient and access the spinal canal is well known. Access to the spinal canal is required in order to remove cerebrospinal fluid from the subarachnoid space surrounding the cauda equina. Typically, the needle is inserted into the skin on the patient's back and is advanced between the lumbar vertebrae to the dura mater. The needle is then advanced into the subarachnoid space.

A typical lumbar puncture (LP) needle enters the subarachnoid space through a hole created in the dura mater. After the LP needle is removed from the subarachnoid space, persistent leakage of cerebrospinal fluid through the hole in the dura mater is a potential contributing factor to post-LP headaches. This persistent leakage which can lead to post-LP headaches occurs from residual tearing of the dura mater during insertion and removal of the needle from the spinal canal. There is also a potential risk of tearing the delicate spinal nerves and tissue during insertion and removal of standard cutting LP needles. Likewise, the act of aspirating or otherwise removing cerebrospinal fluid from the subarachnoid space may create a suction force sufficient to draw nerve roots in the spinal canal into the needle tip, which may potentially contribute to nerve damage and other patient discomfort.

LP needles typically utilize “drip by drip” fluid collection in which a catheter and collection tube remove cerebrospinal fluid one drop at a time relying on the fluid pressure in the subarachnoid space to facilitate drainage. This method requires the patient to remain at the same level relative to the collection tube for an extended period of time; otherwise the pressure differential will change the flow rate of fluid out of the subarachnoid space.

Although it is important to reduce the frequency of post-LP headaches, it is also important to reduce the time required to collect cerebrospinal fluid because the results of testing performed on the removed cerebrospinal fluid are crucial for treatment decision-making. Therefore, if the time required to collect the fluid can be decreased, the results from testing the fluid can be available in a more timely fashion, which will permit earlier treatment decisions and improve hospital outcomes. There is therefore a need for an LP needle that both reduces the potential for post-LP headaches, reduces potential nerve damage, and reduces the time for cerebrospinal fluid collection and testing.

In an effort to reduce the potential for post-LP headaches, some LP needles have been designed with a so-called “atraumatic” or “pencil-point” tip that is not as sharp as the tips on other, more traditional cutting LP needles. Such atraumatic needles appear more likely to spread tissue apart, rather than cutting it. Use of atraumatic needles, however, requires more skill, experience, and time than use of more traditional LP needles. Generally, use of an atraumatic needle involves an “introducer” or cutting needle. After withdrawing the cutting needle from the tissue of the back, the atraumatic needle is located and inserted into the path made by the cutting needle and advanced until the dura mater is penetrated. Since the cutting needle is typically withdrawn prior to insertion of the atraumatic needle, it may be difficult to find the exact path made by the cutting needle. This may require multiple directional adjustments of the atraumatic needle in order to conform to the initial cutting needle path, resulting in multiple needle sticks and patient discomfort.

Accordingly, there is also a need to provide an LP needle that requires only one needle stick to the patient to reduce patient discomfort and injury to local tissues, as well as allowing the practitioner to perform LP procedures safely without requiring an extended time period and extensive expertise.

### SUMMARY OF THE INVENTION

The present invention is directed to an apparatus for removing cerebrospinal fluid. The apparatus includes a first needle having a first end, a second end, and a passage extending between the first and second ends. The first end of the first needle has a handle, and the second end has a cutting tip adapted to penetrate skin and deep tissue. A second needle is slidably disposed within the passage of the first needle. The second needle has a proximal end, a distal end, and a lumen extending between the proximal and distal ends. The proximal end of the second needle has a flange. The distal end of the second needle has an atraumatic tip with a hole pattern in fluid communication with the lumen for removing cerebrospinal fluid. This hole pattern allows for rapid and optimal cerebrospinal fluid extraction via a syringe withdrawal mechanism.

The present invention is also directed to a method for removing cerebrospinal fluid. The method includes providing a first needle having a first end, a second end, and a passage extending between the first and second ends, the first end having a handle and the second end having a cutting tip adapted to penetrate skin. A second needle is provided which is slidably disposed within the passage of the first needle, the second needle having a proximal end, a distal end, and a lumen extending between the proximal and distal ends, the proximal end having a flange, the distal end having an atraumatic tip with a hole pattern in fluid communication with the passage. The skin between adjacent vertebra is penetrated with the cutting tip of the first needle. The atraumatic tip of the second needle is then advanced through the lumen of the larger first needle. The distal end of the second needle is then advanced into the subarachnoid space. Cerebrospinal fluid is removed from the subarachnoid space through the hole patterned tip in the second needle via the syringe withdrawal mechanism.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1A is a front view of a first needle in accordance with the present invention;

FIG. 1B is a side view of the first needle of FIG. 1A;

FIG. 2 is a top view of the first needle of FIG. 1A taken along line 2-2 in FIG. 1B;

FIG. 3A is a front view of a second needle in accordance with the present invention;

FIG. 3B is a sectional view taken along line 3B-3B in FIG. 3A;

FIG. 4 is a top view taken along line 4-4 in FIG. 3A;

FIG. 5 is a front view of an assembled apparatus for removing cerebrospinal fluid in accordance with the present invention;

FIG. 6 is a schematic illustration of a human spinal column;

FIG. 7 is a schematic illustration of the apparatus of FIG. 5 being inserted into the skin of a human at the lumbar region of the spine;

FIG. 8 is an enlarged view of the insertion of FIG. 7;

FIG. 9 is an enlarged top view of the distal end of the first needle of FIG. 8;

FIG. 10 is a sectional view of the distal end of the first needle taken along line 10-10 in FIG. 9;

FIG. 11 is a sectional view of the apparatus inserted between the 4th and 5th lumbar vertebra;

FIG. 12 is a view similar to FIG. 11 illustrating the apparatus in a different position;

FIG. 13A is a sectional view of the apparatus illustrating the removal of cerebrospinal fluid from the subarachnoid space;

FIG. 13B is a sectional view of the apparatus according to an alternative embodiment illustrating the removal of cerebrospinal fluid from the subarachnoid space via a syringe withdrawal mechanism;

FIG. 14 is an enlarged view of a portion of FIG. 13;

FIG. 15 is a front view of an assembled apparatus for removing cerebrospinal fluid in accordance with an alternative embodiment of the present invention;

FIG. 16 is side view of the apparatus of FIG. 15;

FIG. 17 is a schematic illustration of the apparatus of FIG. 15 being inserted into the skin of a human at the lumbar region of the spine; and

FIG. 18 is a sectional view of the apparatus of FIG. 15 illustrating removal of cerebrospinal fluid from the subarachnoid space.

### DETAILED DESCRIPTION

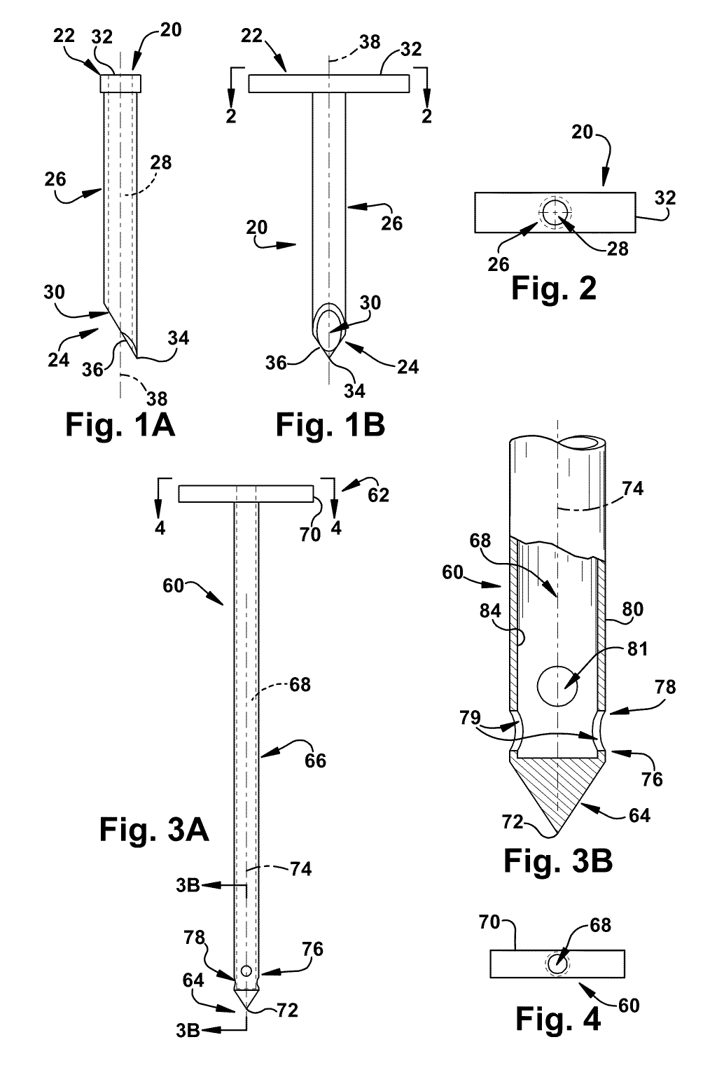

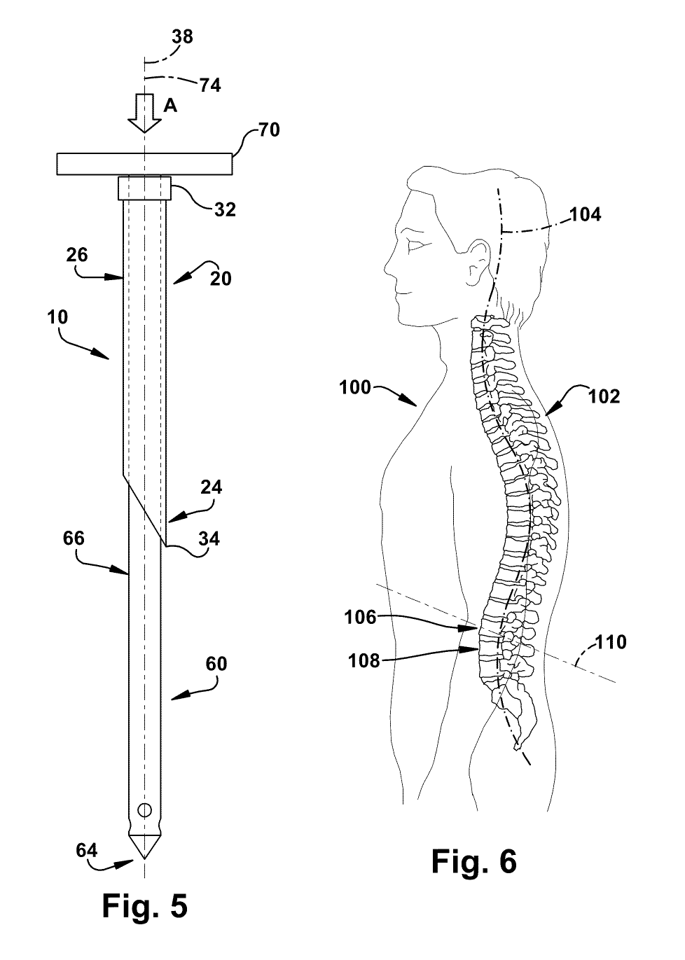

The present invention is directed to a method and apparatus for removing cerebrospinal fluid. As representative of the present invention, a probe 10 (FIG. 5) is illustrated comprising a first needle 20 that extends over a second needle 60. The hollow first needle 20 (FIGS. 1A and 1B) includes a body portion 26 with a first end 22 and a second end 24. The body portion 26 is generally cylindrical in shape and includes a central passage 28 extending the entire length of the body portion. Although the inner and outer circumferential surfaces of the body portion 26 are depicted as being circular (FIG. 2), these surfaces may have any suitable shape. The body portion 26 may be made of any suitable biocompatible metal or polymer, and is strong enough to be advanced through skin and soft tissue without plastic deformation.

The first end 22 of the body portion 26 includes a handle 32. The handle 32 provides the user with a larger surface area with which to grasp and manipulate the first needle 20, as will be hereinafter described. The handle 32 may be rectangular in shape or may be otherwise designed for grasping.

The second end 24 of the body portion 26 includes a cutting tip 34, which allows the user to cut through skin and soft tissue. The second end 24 further includes a central opening 30 (FIGS. 1A and 1B), which communicates with the central passage 28. The central opening 30 and central passage 28 are sized to receive the second needle 60.

The second needle 60 (FIGS. 3A-4) includes a body portion 66 with a proximal end 62 and a distal end 64. The body portion 66 of the second needle 60 is substantially longer than the body portion 26 of the first needle 20. The body portion 66 is generally cylindrical in shape and includes a central lumen 68 extending from the proximal end 62 toward the distal end 64. The distal end 64 of the body portion 66 includes an atraumatic tip 72 that is a non-cutting tip, compared to the cutting tip 34 of the first needle 20. Although the body portion 66 of the second needle 60 is hollow, the tip 72 has a solid construction. The body portion 66 may be made of a suitable biocompatible metal or polymer and is strong enough to be advanced through tissue inside a spinal canal.

The proximal end 62 of the body portion 66 includes a flange 70 similar to the handle 32 of the first needle 20. The flange 70 is rectangular in shape, but may have any shape suitable for grasping by the user. The end of this flange 70 in configured to allow a standard syringe to easily attach for cerebrospinal fluid withdrawal. Although the outer and inner circumferential surfaces 80 and 84 of the body portion 66 are depicted as being circular, these surfaces may have any suitable shape. Regardless of their shapes the body portions 26 and 66 of the first and second needles 20 and 60, respectively, must be formed such that the second needle will fit and slide in the central passage 28 of the first needle. More specifically, a longitudinal central axis 38 of the body portion 26 of the first needle 20 will be co-axial with a longitudinal central axis 74 of the body portion 66 of the second needle 60.

The distal end 64 of the body portion 66 includes a microfilter 76 (FIG. 3B), which provides a path for getting cerebrospinal fluid 118 into the lumen 68 of the second needle 60. The microfilter 76 includes a section of the body portion 66 through which a plurality of openings 78 extend. The openings 78 serve as passages by which cerebrospinal fluid can be removed from within a subarachnoid space 116, through the lumen 68, and out the proximal end 62 of the hollow second needle 60 to a collection reservoir, such as a syringe (not shown). The openings 78 (FIG. 3B) may be circular or elliptical in shape, or otherwise suitably shaped to allow cerebrospinal fluid 118 to pass from the outer surface 80 of the second needle 60 into the lumen 68. The openings 78 may be numbered and spaced about the periphery of the outer surface 80 such that the microfilter 76 provides a 360 degree access area for cerebrospinal fluid to enter without requiring rotation or movement of the second needle 60.

In particular, FIG. 3B illustrates that the plurality of openings 78 include a first pair of diametrically opposed openings 79 located at a first position along the axis 74 and a second pair of diametrically opposed openings 81 located at a second, different, position along the axis 74. The first and second pairs of openings 79, 81 are therefore both radially and longitudinally offset from one another, although it will be understood that the openings 79, 81 may not be offset from one another in the radial and/or longitudinal direction and still provide 360 degrees of access area for cerebrospinal fluid removal.

Each of the first pair of openings 79 and each of the second pair of openings 81 extends from the outer surface 80 of the body portion 66 to the inner surface 84 and communicates with the lumen 68. Although one pair of first openings 79 and one pair of second openings 81 are shown in FIG. 3B, it will be understood that more or fewer of each pair of openings may be used. It will also be understood that an odd number of openings 78 may be used in accordance with the present invention.

Alternatively, the openings 78 can constitute one or more rings of closely spaced openings (not shown) extending around the entire periphery of the second needle 60. In any case, the aforementioned patterns for the openings 78 allows continuous cerebrospinal fluid flow through the microfilter 76 even if some openings become blocked by local tissue.

As shown in FIG. 5, to assemble the probe 10, the distal end 64 of the second needle 60 is inserted into the central passage 28 of the first needle 20 at the first end 22 and pushed towards the second end 24 of the first needle. The insertion direction is indicated by arrow “A” in FIG. 5. Since the first needle 20 and the second needle 60 are not secured relative to each other, the body portion 26 of the first needle is capable of sliding relative to the body portion 66 of the second needle.

FIG. 6 illustrates the spine 102 of a patient 100 into which the probe 10 may be inserted. An axis 104 extends along the spine 102 and follows the contour of the spine. The probe 10 may be inserted into the patient 100 along an insertion axis 110 which extends transverse to, and through, the axis 104 of the spine 102. The insertion axis 110 may be positioned anywhere along the spine 102. For example, the insertion axis 110 may extend between the L4 and L5 vertebra 106 and 108 of the spine 102.

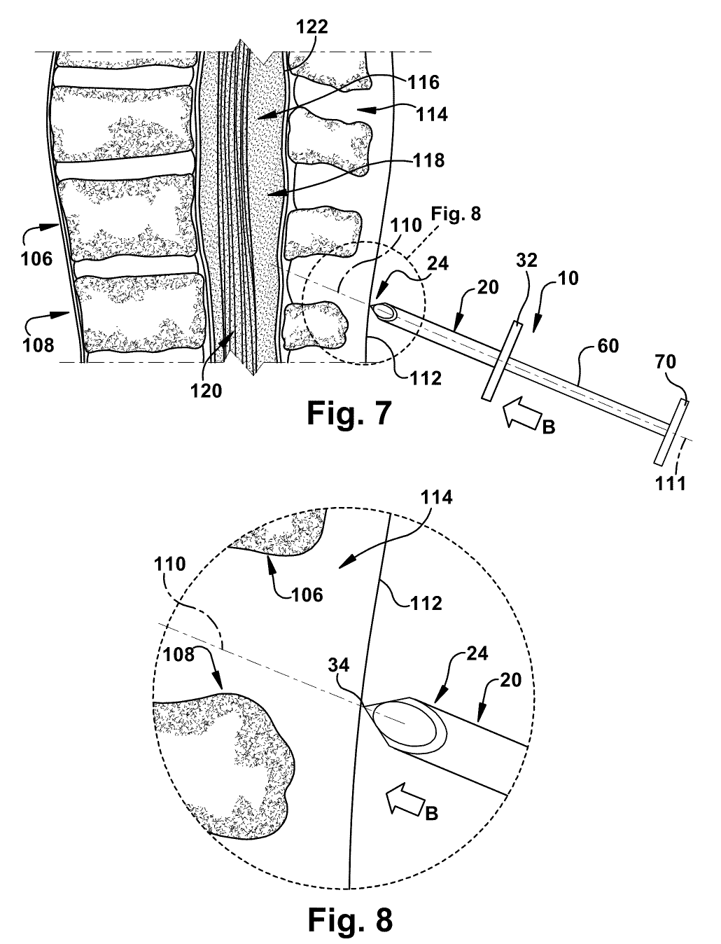

As shown in FIGS. 7-8, once the first needle 20 has been slidably mounted on the second needle 60, the first needle is moved relative to the second needle, as illustrated by arrow “B”, until the second end 24 of the first needle is advanced beyond the distal end 64 of the second needle. The second end 24 of the first needle 20 is then placed in contact with the skin 112 on the back of the patient 100. The probe 10 is oriented such that a longitudinal central axis 111 of the probe 10 is co-axial with the desired insertion axis 110. In the present example, the insertion axis 110 extends through the skin 112 and soft tissue 114 of the patient 100 and into the subarachnoid space 116, which surrounds the cauda equina 120 and in which cerebrospinal fluid 118 is located. It is desirable to maintain a pathway transverse to the spine 102 along the insertion axis 110 for positioning the probe 10 to access the subarachnoid space 116 without the interference of vertebra. Although the L4 and L5 vertebra 106 and 108 of the spine 102 are depicted, it is understood that the probe 10 may be inserted between any vertebra (i.e., cervical, thoracic or lumbar) for the removal of cerebrospinal fluid 118.

As shown in FIG. 8, the configuration and orientation of the probe 10 is such that the cutting tip 34 of the first needle 20 acts as the leading edge for the probe. The tip 34 is driven in the direction of arrow B along the insertion axis 110 to penetrate the skin 112 and soft tissue 114 surrounding the vertebra 106, 108. Penetrating the skin 112 and soft tissue 114 with the tip 34 of the first needle 20 is advantageous because the tip 72 of the second needle 60 is blunter than the tip 34 of the first needle and will not as easily puncture the skin. The skin 112 and soft tissue 114 penetration by the first needle 20 will facilitate subsequent passage of the second needle 60 through the soft tissue and ultimately between the vertebra 106, 108 to access the subarachnoid space 116 for the collection of cerebrospinal fluid 118.

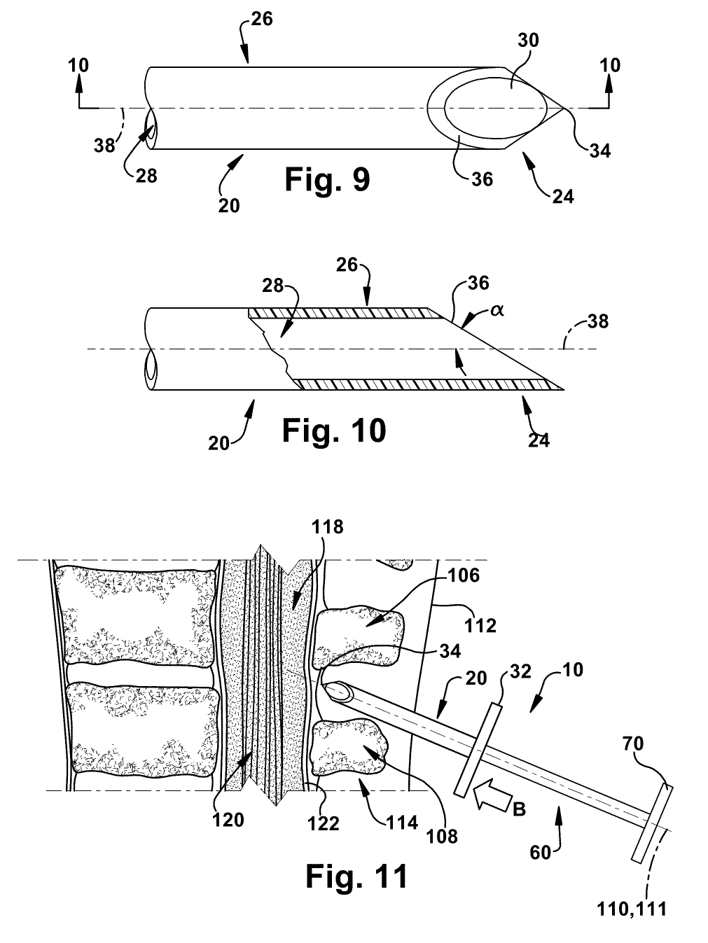

FIGS. 9-10 depict detailed drawings of the cutting tip 34 of the first needle 20. The cutting tip 34 includes a surface 36 formed at an angle α relative to the longitudinal central axis 38 of the first needle. The surface 36 defines the opening 30 as having an oblong profile and communicating with the central passage 38 of the first needle 20. As noted, the shape of the tip 34 allows the user to drive the tip through skin 112 and soft tissue 114. The angled surface 36 assists the tip 34 in cutting through the skin 112 and the soft tissue 114. Furthermore, the surface 36 is configured such that the opening 30 is unobstructed to allow the distal end 64 of the second needle 60 to advance through and beyond the opening. Those having ordinary skill, however, will appreciate that the surface 36 and/or the opening 30 can have alternative constructions within the spirit of the present invention.

As shown in FIG. 11, the skin 112 is penetrated by applying a force to the handle 32 on the first needle 20 along the insertion axis 110 in the direction of arrow B to force the cutting tip 34 of the first needle through the skin. Once the skin 112 has been penetrated, advancement of the second end 24 of the first needle 20 through the soft tissue 114 is facilitated by the cutting tip 34. The second end 24 of the first needle 20 is advanced between the L4 vertebra 106 and the L5 vertebra 108 until the tip 34 of the first needle is close to the dura mater 122. The body portion 26 of the first needle 20 may be provided with indicia (not shown) indicating the penetration depth of the first needle into the soft tissue 114.

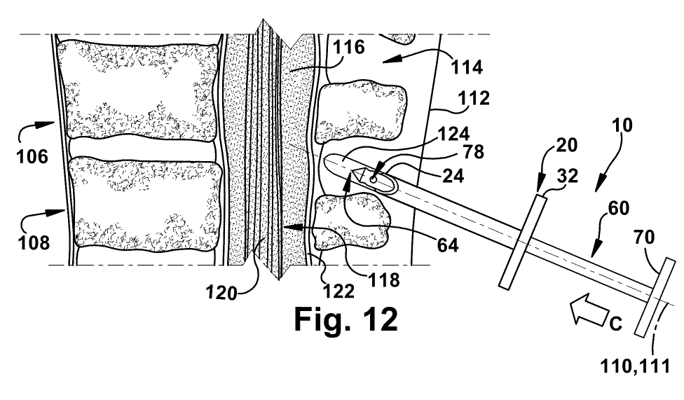

FIG. 12 illustrates that the advancement of the first needle 20 into the soft tissue 114 creates a channel 124 through the tissue along the insertion axis 110. Based on the configuration of the first needle 20 and the second needle 60, the channel 124 created by the larger-profiled first needle allows the second needle to be advanced easily through the shaft and into the dura mater 122 once the distal end 64 of the second needle has been advanced through the passage 28 and beyond the opening 30 of the first needle.

To advance the distal end 64 of the second needle 60 beyond the second end 24 of the first needle 20 and into the channel 124, as shown in FIG. 12, a force is applied in the direction indicated by arrow “C” to the flange 70 on the second needle along the central axis 111 of the probe and co-axial with the insertion axis 110 while the first needle 20 is held stationary. This causes the second needle 60 to slide within and relative to the passage 28 of the first needle 20 so that the distal end 64 of the second needle and the second end 24 of the first needle effectively switch places along the insertion axis 110. Care must be taken, however, while the first needle 20 and the second needle 60 are sliding relative to one another, as either the second end 24 of the first needle or the distal end 64 of the second needle should remain in the channel 124 so that the probe 10 maintains proper alignment along the insertion axis 110. This prevents the need to re-insert the probe 10 into the tissue 114 and, thus, the channel 124, which helps minimize patient discomfort and procedure time.

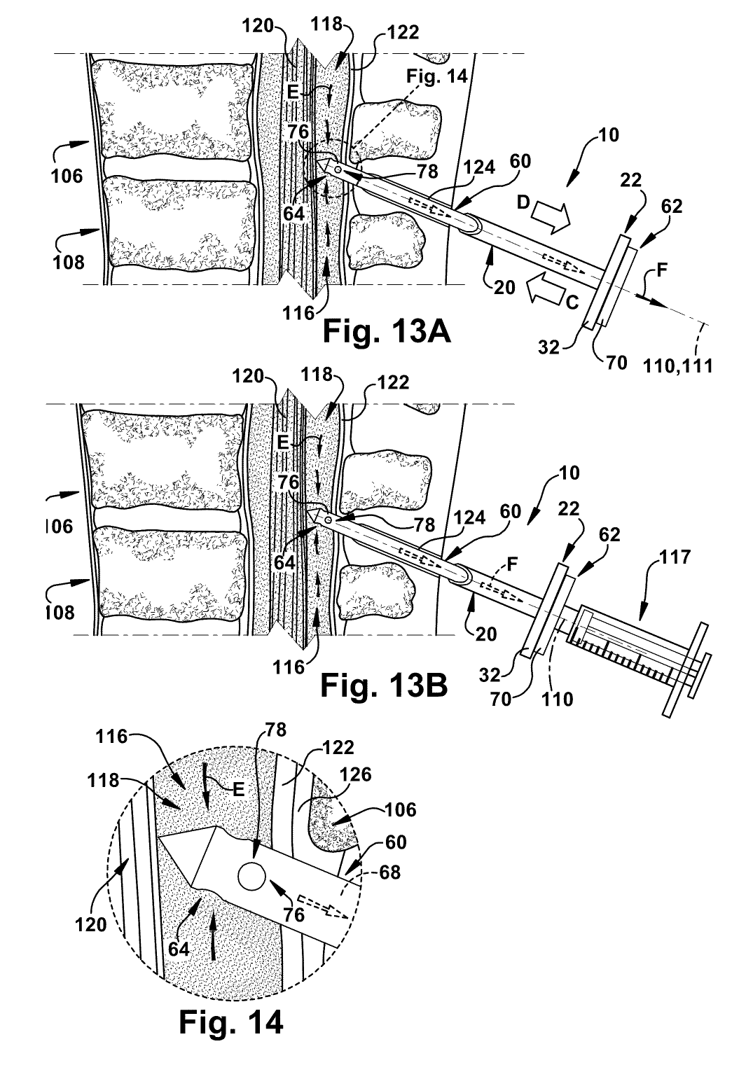

The second needle 60 is sufficiently advanced so that the distal end 64 of the second needle is in proximity with the dura mater 122 (FIG. 13A). Due to the softer nature of the dura mater 122, continued advancement in the direction of arrow C of the flange 70 of the second needle 60 drives the atraumatic tip 72 of the second needle through the dura mater and into the subarachnoid space 116 surrounding the cauda equina 120. The tip 72 of the second needle 60 is advanced until the microfilter 76 in the distal end 64 of the second needle is disposed within the subarachnoid space 116. Once the second needle 60 has been properly located relative to the dura mater 122, the first needle 20 may be retracted back away from the dura mater by pulling the handle 32 on the first needle in the direction indicated by arrow “D” along the insertion axis 110. The first needle 20 can be retracted out of the patient until the handle 32 of the first needle abuts the flange 70 of the second needle 60. It will be understood, however, that it may be desirous to maintain the first needle 20 within the channel 124 to ensure proper alignment and placement of the second needle 60 relative to the dura mater 122 (not shown).

Since the proximal end 62 of the second needle 60 is in fluid communication with the microfilter 76 via the lumen 68, the placement of the microfilter within the subarachnoid space 116 allows the removal of cerebrospinal fluid 118 to begin. One way to remove the cerebrospinal fluid 118 is via the traditional drip method, which utilizes gravity to remove the cerebrospinal fluid from the subarachnoid space 116 one drop at a time. Alternatively, with this LP system, a suction device such as a standard 3 cc syringe 117 (FIG. 13B) in fluid communication with the proximal end 62 of the second needle 60 can be used to apply suction to the lumen 68 of the second needle and pull cerebrospinal fluid 118 into the microfilter 76 at the distal end 64 of the second needle. By using a syringe 117, the rate of cerebrospinal fluid 118 entering the probe 10 is greatly increased, thereby decreasing the time spent doing the procedure. As discussed, the configuration of the microfilter 76 allows cerebrospinal fluid 118 to be aspirated through the openings 78 and into the lumen 68.

As indicated by arrow E (FIGS. 13A-14), the cerebrospinal fluid 118 is drawn through the openings 78 and into the distal end 64 of the lumen 68, travels through the body portion 66 of the second needle 60, and is drawn out through the proximal end 62 of the second needle, as indicated by arrow F, into a reservoir or other storage container for preservation and collection. The orientation of the microfilter 76 allows collection of cerebrospinal fluid 118 around the entire 360 degree circumference of the distal end 64 of the second needle 60. Such a construction is advantageous in that it increases the rate at which the cerebrospinal fluid 118 can be collected. This construction may also help avoid clogging of the microfilter 76 since there is 360 degree availability for entrance of the cerebrospinal fluid 118 into the probe 10.

The configuration and opening 78 size of the microfilter 76 further allows cerebrospinal fluid 118 to enter the lumen 68 of the second needle 60 quickly via syringe withdrawal while preventing spinal nerves or other spinal tissue from being inadvertently drawn into the lumen and damaged. Therefore, a relatively large amount of cerebrospinal fluid 118 can be collected while the second needle 60 is disposed along a single line within the subarachnoid space 116 without movement of the tip 72 of the second needle.

When the syringe 117 is full of cerebrospinal fluid 118, the syringe is removed from the proximal end 62 of the second needle 60 and the cerebrospinal fluid is ejected into a cerebrospinal fluid collection tube (not shown), as found in a standard lumbar puncture kit. This sequence is continued until the desired amount of cerebrospinal fluid 118 has been collected. Following the collection of cerebrospinal fluid 118, the second needle 60 is removed from the subarachnoid space 116, and subsequently the soft tissue 114 and skin 112 along with the first needle 20 by pulling the handle 32 on the first needle along the insertion axis 110 indicated by arrow D (see FIG. 13A). Since the handle 32 on the first needle 20 abuts the flange 70 on the second needle 60, and the first needle is disposed over the second needle, pulling the handle on the first needle will cause both the first needle and the second needle to retract out of the patient for complete removal of the probe 10.

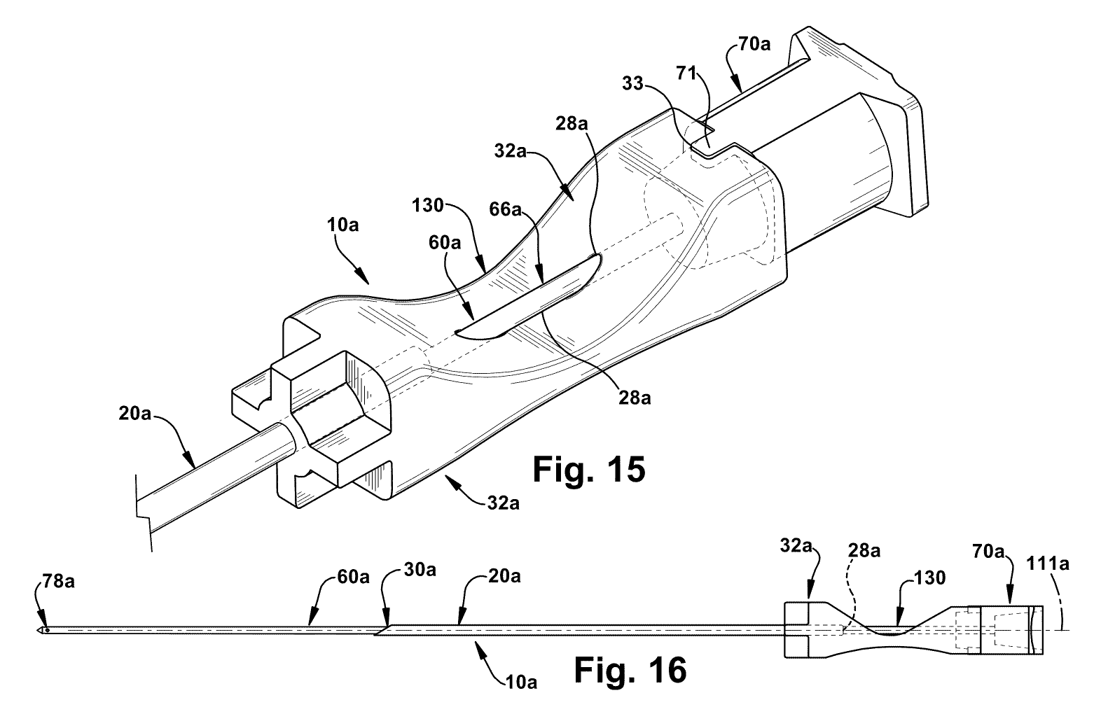

A second embodiment of the present invention is illustrated in FIGS. 15-18. The second embodiment of the invention is similar to the first embodiment of the invention illustrated in FIGS. 1-14. Accordingly, numerals similar to those of FIGS. 1-14 will be utilized in FIGS. 15-18 to identify similar components, the suffix letter “a” being associated with the numerals of FIGS. 15-18 to avoid confusion. The probe 10a (FIGS. 15-18) of the second embodiment is identical to the probe 10 (FIGS. 1-14), except that the handle 32a on the first needle 20a and the flange 70a on the second needle 60a in the second embodiment have different configurations than the handle 32 on the first needle 20 and the flange 70 on the second needle 60 of the first embodiment. Those skilled in the art, however, will appreciate that the handle 32a and the flange 70a illustrated in the needle 10a of the second embodiment could also be implemented into the needle 10 of the first embodiment.

FIGS. 15-16 illustrate that the handle 32a on the first needle 20a is designed to allow for optimal stabilization of the underlying second needle 60a as the soft tissue of the back of the patient is penetrated by the first needle 20a. This stabilization may prevent the second needle 60a from being pushed away from the tissue during penetration by the first needle 20a. The flange 70a on the second needle 60a includes a projection 71 for engaging a recess 33 in the handle 32a of the first needle 20a when the body portion 66a of the second needle 60a is disposed within the central passage 28a of the first needle 20a. Other engagement means, however, between the handle 32a on the first needle 20a and the flange 70a on the second needle 60a, such as threads, clips or hooks are also contemplated.

Grip and stabilization of the first needle 20a is facilitated by providing the handle 32a on the first needle 20a with a generally concave shape. Furthermore, the handle 32a on the first needle 20a is configured such that the user can engage the body portion 66a of the second needle 60a when the body portion 66a of the second needle 60a is disposed within the central passage 28a of the first needle 20a. In other words, the user is able to engage both the handle 32a on the first needle 20a and the body portion 66a of the second needle 60a simultaneously. The handle 32a on the first needle 20a may include, for example, a lateral opening 130 that allows the user to engage both the handle on the first needle and the body portion 66a of the second needle 60a simultaneously.

This construction is advantageous in that it allows the user to support and stabilize both needles 20a, 60a at the same time, regardless of the relative positioning between the handle 32a on the first needle and the handle 70a on the second needle. In other words, the first needle 20a can be slid along the second needle 60a along the axis 111a to any longitudinal position until the user wishes to stabilize both needles at the same time. The user would then grasp the handle 32a on the first needle 20a while simultaneously using the lateral opening 130 in the handle 32a to apply force to the body portion 66a of the second needle 60a, thereby rigidly coupling both needles together.

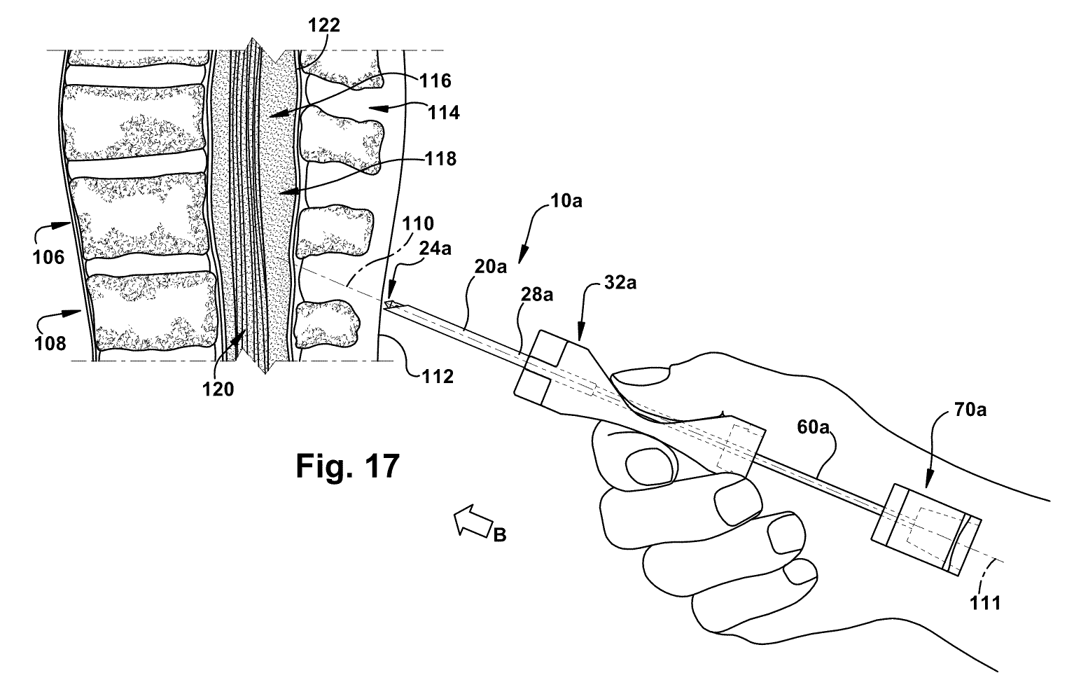

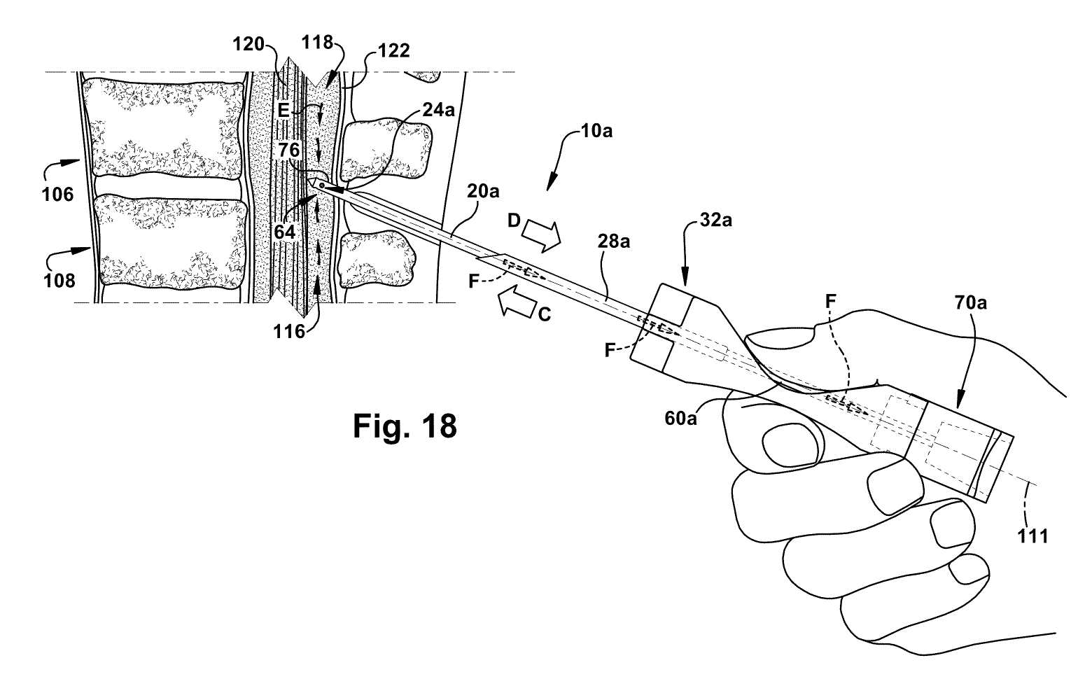

This would allow the user to, for example, stabilize both needles 20a, 60a with the tip of the first needle 20a aligned with the tip of the second needle 60a such that the user can penetrate the tissue of the back with both needles 20a, 60a simultaneously, as shown in FIG. 17. By stabilizing both needles 20, 60a simultaneously, this construction is advantageous in that allows both needles to enter the soft tissue 114 at the same time in a single, stable insertion. The handle 32a construction of the probe 10a would also allow the user to stabilize both needles 20a, 60a while the user is retracting either just the second needle or both the first needle and the second needle from the patient following the removal of cerebrospinal fluid 118, as shown in FIG. 18.

Furthermore, by stabilizing both needles 20a, 60a simultaneously during penetration, the second needle 60a is prevented from pushing backwards away from the patient's back as the skin is penetrated. Since it is desirous to align the tips of both the first needle 20a and second needle 60a during this penetration, the user can grasp the handle 32a of the first needle to stabilize both needles and ensure proper tip alignment. This will allow for more accurate aiming of the probe 10a within the soft tissue 114 and, thus, more accurate alignment of the probe with the desired insertion axis 110a, as opposed to merely holding the handle 32a of the first needle 20a and not the body portion 66a of the second needle 60a.

It will be appreciated that when the body portion 66a of the second needle 60 is being stabilized, the body portion is not constricted or deformed, thereby maintaining fluid flow through the body portion. Therefore, the stabilization of both needles 20a, 60a during subsequent removal of the cerebrospinal fluid 118 through the openings 78a does not restrict or hinder the flow of the cerebrospinal fluid through the second needle and into the reservoir, storage unit or syringe (not shown).

As with the openings 78 in the probe 10, the openings 78a in the probe 10a allow for 360 degrees of access area for cerebrospinal fluid 118 removal. The openings 78a may exhibit the same construction as the openings 78 such as, for example, multiple pairs of diametrically opposed and longitudinally offset openings (not shown) or otherwise any configuration that allows for 360 degrees of access area.

From the above description of the invention, those skilled in the art will perceive improvements, changes and modifications. Such improvements, changes and modifications within the skill of the art are intended to be covered by the appended claims.

Having described the invention, the following is claimed:

1. An apparatus for removing cerebrospinal fluid, the apparatus comprising:
a first needle having a first end, a second end and a passage extending between the first and second ends, the first end having a handle, and the second end having a cutting tip adapted to penetrate skin; and
a second needle slidably disposed within the passage of the first needle, the second needle having a proximal end, a distal end, and a lumen extending between the proximal and distal ends, the proximal end having a flange, the distal end having an atraumatic tip and a microfilter in fluid communication with the lumen for removing cerebrospinal fluid.

2. The apparatus of claim 1, wherein the second needle includes an outer surface, the microfilter comprising a plurality of openings disposed around the periphery of the distal end of the second needle and extending from the outer surface to the lumen.

3. The apparatus of claim 2, wherein the openings provide a 360 degree access area for cerebrospinal fluid to enter the lumen.

4. The apparatus of claim 3, wherein the openings comprise a first pair of openings and a second pair of openings radially and longitudinally offset from the first pair of openings relative to a longitudinal axis of the second needle.

5. The apparatus of claim 1, wherein the atraumatic tip of the second needle is solid.

6. The apparatus of claim 1, wherein the cutting tip on the first needle includes an angled surface that forms an oblong opening communicating with the passage of the first needle.

7. The apparatus of claim 1, wherein the handle of first needle includes a lateral opening such that the first needle and the second needle can be engaged simultaneously to stabilize the first and second needles during skin penetration.

8. The apparatus of claim 7, wherein the handle of the first needle is concave.

9. The apparatus of claim 1, wherein the handle of the first needle and the handle of the second needle include means for engaging the handle of the first needle and the handle of the second needle.

10. The apparatus of claim 1 further comprising a syringe for applying suction to the lumen of the second needle to remove the cerebrospinal fluid.

11. A method for removing cerebrospinal fluid, the method comprising the steps of:
providing a first needle having a first end, a second end and a passage extending between the first and second ends, the first end having a handle and the second end having a cutting tip adapted to penetrate skin;
providing a second needle slidably disposed within the passage of the first needle, the second needle having a proximal end, a distal end, and a lumen extending between the proximal and distal ends, the proximal end having a handle, the distal end having an atraumatic tip having a microfilter in fluid communication with the passage;
penetrating the skin between adjacent vertebra with the pointed tip of the first needle;
exposing the atraumatic tip of the second needle;
advancing the distal end of the second needle into the subarachnoid space; and
removing cerebrospinal fluid from the subarachnoid space through the microfilter in the second needle via syringe withdrawal collection.

12. The method of claim 11, wherein the step of penetrating the skin between adjacent vertebra includes simultaneously holding the first needle and the second needle through an opening in the handle of the first needle.

13. The method of claim 11, wherein the step of removing cerebrospinal fluid comprises removing cerebrospinal fluid over a 360 degree access area of the second needle while the second needle is stationary.
